(19) United States
(12) Patent Application Publication
(10) Pub. No.: US 2026/0069743 A1
(43) Pub. Date: Mar. 12, 2026

(54) PIEZOELECTRIC BONE CEMENTS AND CELL CULTURE DISHES

(71) Applicant: Joon Bu Park, Las Vegas, NV (US)

(72) Inventor: Joon Bu Park, Las Vegas, NV (US)

(21) Appl. No.: 19/388,614

(22) Filed: Nov. 13, 2025

Related U.S. Application Data
(62) Division of application No. 17/479,591, filed on Sep. 20, 2021.

(51) Int. Cl. A61L 24/00; A61C 8/00; A61F 2/28

(52) U.S. Cl. CPC A61L 24/0089; A61C 8/0016; A61F 2/28; A61L 24/0084; A61F 2310/00353; A61L 2430/02; A61L 2430/12

(57) ABSTRACT

A bone cement includes a liquid component including a monomer configured to polymerize upon curing of the bone cement; and a solid component dispersed in the liquid component, the solid component including a powder of a polymer; an initiator; and a powder of a piezoelectric ceramic. A cell culture dish includes a base; and walls connected to the base to define an interior of the cell culture dish, wherein at least a portion of an interior surface of the base includes a piezoelectric material.

### CROSS-REFERENCE TO RELATED APPLICATIONS

This application is a divisional application of and claims the benefit of priority to U.S. application Ser. No. 17/479,591, filed on Sep. 20, 2021, the contents of which are hereby incorporated by reference.

### BACKGROUND

The present disclosure relates generally to materials for bone cements and cell culture dishes.

### SUMMARY

We describe here bone cement and cell culture dishes that include materials having piezoelectric properties (“piezoelectric materials”). Piezoelectric materials accumulate electric charge in response to an applied mechanical stress, and also generate mechanical strain in response to an applied electric field. For example, some piezoelectric materials may generate measurable electric charge when their static structure is deformed by about 0.1% of the original dimension. Some piezoelectric materials may change about 0.1% of their static dimension when an external electric field is applied to the material. Piezoelectric materials in bone cement can have advantageous X-ray contrast characteristics and can have a higher density than traditional materials, meaning that less need be used to achieve a desired level of contrast. Additionally, the presence of piezoelectric materials in bone cement may promote bone growth, improving healing. Piezoelectric materials in cell culture dishes also can stimulate cell and tissue growth.

In an aspect, a bone cement includes a liquid component including a monomer configured to polymerize upon curing of the bone cement; and a solid component dispersed in the liquid component, the solid component including a powder of a polymer; an initiator; and a powder of a piezoelectric ceramic.

Embodiments of the bone cement can include one or any combination of two or more of the following features.

The piezoelectric ceramic includes at least one of barium titanate or hydroxyapatite.

The powder of the piezoelectric ceramic includes between 5 and 15 weight percent of the solid component.

The powder of the polymer includes a powder of at least one of polymethyl methacrylate (PMMA), methyl methacrylate-styrene copolymer, polystyrene, or methacrylate copolymer.

The solid component includes between 10 and 20 weight percent of a powder of PMMA and between 70 and 80 weight percent of a powder of methyl methacrylate-styrene copolymer.

The initiator includes at least one of dibenzoyl peroxide or benzoyl peroxide.

The monomer includes methyl methacrylate.

The liquid component includes an accelerator, such as at least one of N,N,-dimethyl-p-toluidine or dimethyl para-toluidine.

The liquid component includes a stabilizer, such as hydroquinone.

The liquid component includes between 95 and 99 volume percent of the monomer, wherein the monomer includes methyl methacrylate monomer; between 1 and 4 volume percent of an accelerator; and a stabilizer. The solid component includes between 85 and 95 weight percent of the powder of the polymer, wherein the powder of the polymer includes a powder of polymethyl methacrylate (PMMA), methyl methacrylate-styrene copolymer, or both; and between 5 and 15 weight percent of the powder of the piezoelectric ceramic, wherein the piezoelectric ceramic includes barium titanate.

In an aspect, a method of using bone cement to affix an implant to bone includes mixing the bone cement, including dispersing a solid component of the bone cement in a liquid component of the bone cement, wherein the liquid component of the bone cement includes a monomer configured to polymerize upon curing of the bone cement, and wherein the solid component of the bone cement includes a powder of a polymer, an initiator, and a powder of a piezoelectric ceramic; applying the mixed bone cement to a surface of the bone; attaching the implant to the bone cement on the surface of the bone; and curing the bone cement to affix the implant to the bone.

Embodiments of the method can include one or any combination of two or more of the following features.

The method includes applying a second powder of a second piezoelectric ceramic to the surface of the bone, and in which applying the mixed bone cement includes applying the mixed bone cement to the surface of the bone having the second powder applied thereon.

The second powder of the second piezoelectric ceramic includes a powder of a composite of barium titanate and hydroxyapatite.

The method includes coating the second powder of the second piezoelectric ceramic with bone stem cells.

The piezoelectric ceramic includes at least one of barium titanate or hydroxyapatite.

The powder of the polymer includes a powder of at least one of polymethyl methacrylate, methyl methacrylate-styrene copolymer, polystyrene, or methacrylate copolymer.

The monomer includes methyl methacrylate.

The liquid component includes an accelerator.

The liquid component includes a stabilizer.

The liquid component includes between 95 and 99 volume percent of the monomer, wherein the monomer includes methyl methacrylate monomer; between 1 and 4 volume percent of an accelerator; and a stabilizer. The solid component includes: between 85 and 95 weight percent of the powder of the polymer, wherein the powder of the polymer includes a powder of polymethyl methacrylate (PMMA), methyl methacrylate-styrene copolymer, or both; between 5 and 15 weight percent of the powder of the piezoelectric ceramic, wherein the piezoelectric ceramic includes barium titanate.

In an aspect, a bone structure includes a bone; a bone implant; and bone cement disposed between the bone and the bone implant and affixing the bone to the bone implant, in which the bone cement includes a polymeric matrix and a particles of piezoelectric ceramic dispersed in the polymeric matrix.

Embodiments of the bone structure can include one or any combination of two or more of the following features.

The piezoelectric ceramic includes at least one of: barium titanate and hydroxyapatite.

The polymeric matrix includes at least one of polymethyl methacrylate, methyl methacrylate-styrene copolymer, polystyrene, or methacrylate copolymer.

The bone structure includes a layer of a powder of piezoelectric ceramic disposed on a surface of the bone between the bone and the bone cement.

The powder of piezoelectric ceramic includes a coating of bone stem cells.

The powder of piezoelectric ceramic includes a composite of barium titanate and hydroxyapatite.

The implant includes an orthopedic implant.

The implant includes a dental implant.

In an aspect, a cell culture dish includes a base; and walls connected to the base to define an interior of the cell culture dish, wherein at least a portion of an interior surface of the base includes a piezoelectric material.

Embodiments of the cell culture dish can include one or any combination of two or more of the following features.

The piezoelectric material is quartz.

The piezoelectric material is barium titanate.

The piezoelectric material is polyvinylidene fluoride.

The interior surface of the base is textured to promote cell growth.

The cell culture dish includes a coating of the piezoelectric material disposed on the base, and wherein the coating forms the interior surface of the base.

The piezoelectric material includes a negative Poisson's ratio (NPR) material, in which the NPR material has a Poisson's ratio of between 0 and −1.

The NPR material includes microspheres of the piezoelectric material.

The NPR material includes microtubules of the piezoelectric material.

In an aspect, a method of making a cell culture dish includes forming at least a portion of an interior surface of a base of a cell culture dish from the NPR material; wherein the cell culture dish includes the base and walls connected to the base to define an interior of the cell culture dish.

Embodiments of the method can include one or any combination of two or more of the following features.

The piezoelectric material is quartz.

The piezoelectric material is barium titanate.

The piezoelectric material is polyvinylidene fluoride.

The method includes forming the piezoelectric material into a negative Poisson's ratio (NPR) material, in which the NPR material has a Poisson's ratio of between 0 and −1, and in which the portion of the interior surface of the base of the cell culture dish is formed of the piezoelectric NPR material.

Forming the at least portion of the interior surface from the piezoelectric material includes disposing a coating of the piezoelectric NPR material on the base such that the coating forms the interior surface of the base.

Forming the piezoelectric material into an NPR material includes forming microspheres of the piezoelectric material.

Forming the piezoelectric material into an NPR material includes forming microtubules of the piezoelectric material.

The method includes texturing the interior surface of the base to promote cell growth.

Other implementations are within the scope of the claims.

### DESCRIPTION OF DRAWINGS

FIG. 1 is a diagram of an orthopedic implant affixed to a bone with piezoelectric bone cement.

FIG. 2 is a diagram of a dental implant affixed with piezoelectric bone cement.

FIG. 3A is a diagram of a piezoelectric bone cement applied to a polymeric implant.

FIG. 3B is a diagram of multiple layers of material applied to a bone surface.

FIG. 4 is a flow chart of a method of using a piezoelectric bone cement.

FIG. 5 is a diagram of a cell culture dish.

FIG. 6 is an illustration of materials with negative and positive Poisson's ratios.

FIG. 7 is a diagram of a method of making an NPR material.

### DETAILED DESCRIPTION

We describe here bone cement and cell culture dishes that include materials having piezoelectric properties (“piezoelectric materials”). Piezoelectric materials accumulate electric charge in response to an applied mechanical stress, and also generate mechanical strain in response to an applied electric field. For example, some piezoelectric materials may generate measurable electric charge when their static structure is deformed by about 0.1% of the original dimension. Some piezoelectric materials may change about 0.1% of their static dimension when an external electric field is applied to the material. Piezoelectric materials in bone cement can have advantageous X-ray contrast characteristics and can have a higher density than traditional materials, meaning that less need be used to achieve a desired level of contrast. Additionally, the presence of piezoelectric materials in bone cement may promote bone growth, improving healing. Piezoelectric materials in cell culture dishes also can stimulate cell and tissue growth.

Bone cements are materials that are used to affix an object, such as a prosthetic implant (e.g., an orthopedic implant or a dental implant), to a bone. For example, bone cements are used in joint replacements to affix an artificial joint to surrounding bone. Generally, a bone cement fills free space between the prosthetic implant and the bone, creating a close mechanical connection between the bone cement and both the bone and the prosthetic implant. This close connection enables the bone cement to evenly buffer the forces acting against the bone and distributes stresses and interfacial strain energy.

Bone cement is obtained by mixing a solid phase and a liquid phase. The solid phase includes a polymer powder, such as an acrylic polymer (e.g., polymethyl methacrylate (PMMA), methyl methacrylate-styrene copolymer, polystyrene, methacrylate copolymer, or combinations thereof). The liquid phase includes a liquid monomer (e.g., methyl methacrylate) that, upon mixing, polymerizes around the solid polymer particles to form the solid bone cement. The liquid phase can include additional components other than the liquid monomer. For instance, the liquid phase can include an accelerator, such as N, N-dimethyl para-toluidine or dimethyl para-toluidine, to facilitate polymerization; and a stabilizer, such as hydroquinone, to help prevent premature polymerization. The solid phase also can include additional components other than the polymer powder. For instance, the solid phase can include an initiator, such as benzoyl peroxide, that acts an initiator of polymerization. In typical bone cements, the solid phase can include a contrast agent, e.g., a radiopaque component such as zirconium dioxide (ZrO₂) or barium sulfate (BaSO₄) to make the bone cement visible in X-ray or other types of imaging-rays. Other components, such as antibiotics, can be included in the solid phase, the liquid phase, or both. Once cured, a bone cement is a solid polymer matrix in which additives, such as the contrast agent or antibiotics, are dispersed.

The solid phase of the bone cements described here includes powders of piezoelectric materials, such as piezoelectric ceramics (e.g., barium titanate (BaTiO₃) or hydroxyapatite (HA)) in addition to or in place of typical contrast agents. The presence of piezoelectric materials in bone cement can provide good contrast, e.g., X-ray contrast characteristics. In addition, piezoelectric materials often have a higher density than non-piezoelectric materials (e.g., 6.0 g/cm³ for piezoelectric barium titanate versus 4.5 g/cm³ for barium sulfate), meaning that for two bone cements with equal amounts of piezoelectric material and typical contrast agent, a better contrast can be achieved for the piezoelectric bone cement. Similarly, less piezoelectric material need be used than typical contrast agent to achieve a substantially equal contrast.

The inclusion of piezoelectric materials in bone cement can also facilitate attachment of the bone cement to the surface of a bone, and for promoting growth of bone cells. The mechanical stress acting on a piezoelectric bone cement that is disposed between an implant and a bone induces generation of electrical energy in the bone cement. The resulting formation of electric dipoles in the bone cement attracts bone cells (e.g., osteoblasts) that are capable of building bone structures. This process complements the piezoelectric effect that is exhibited by bones themselves and that can also lead to bone growth.

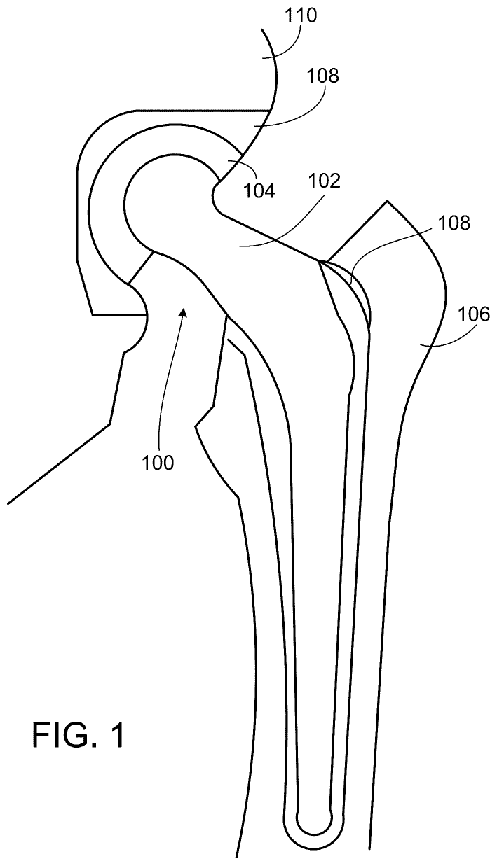

Referring to FIG. 1, bone cement can fasten an orthopedic implant (e.g., a prosthesis) to a bone. FIG. 1 illustrates a hip implant 100. The hip implant 100 includes a metal (e.g., steel, titanium, cobalt, etc.) piece 102 and a plastic (e.g., polyethylene) piece 104. Generally, the plastic piece 104 acts as a socket for the metal piece 102 to act as a joint in the body. In the illustrated example, the plastic piece 104 is acting as the hip socket, and the metal piece 104 is acting as the hip. The metal piece 102 of the implant 100 is attached to a femur 106 of a patient via bone cement 108. The bone cement 108 fastens the implant 100 to the femur 106. For example, the bone cement 108 can applied to the implant 100 and/or the femur 106 as a paste to affix the implant 100 to the femur 106 once the implant is into the patient's body. Similarly, the plastic piece 104 is attached to a pelvis 110 of a patient via bone cement 108. The bone cement affixes the plastic piece 104 to the pelvis 110.

The bone cement 108 is formed of a solid phase including a polymer powder (e.g., PMMA, methyl methacrylate-styrene copolymer, polystyrene, methacrylate copolymers) mixed with a liquid phase including a liquid monomer (e.g., methyl methacrylate). The bone cement 108 can also include one or more additional components as described above, including an initiator, an accelerator, a stabilizer, an antibiotic, or other suitable additional components. The bone cement 108 also includes a piezoelectric material (e.g., barium titanate (BaTiO₃) or hydroxyapatite (HA)). When the bone cement is set, the bone cement includes particles of the piezoelectric material dispersed in a polymer matrix. A bone cement including a piezoelectric material is referred to here as a piezoelectric bone cement.

The piezoelectric materials in the bone cement provide advantageous x-ray contrast characteristics. For example, piezoelectric materials in the bone cement 108 can have a higher contrast than typical contrast agents such as ZrO₂ or BaSO₄. Additionally, some piezoelectric materials (e.g., BaTiO₃, HA) have a higher density than typical, non-piezoelectric contrast agents. Therefore, less piezoelectric ceramic powder can be used for an equal or better contrast during imaging (e.g., X-ray imaging) as compared to typical contrast agents used in bone cements. Additionally, piezoelectric materials can be advantageous for affixing the bone cement to the surface of a bone (e.g., femur 106 or pelvis 108) because bones themselves exhibit a piezoelectric effect, which enables the piezoelectric materials in the bone cement to fasten the implant to the bone more securely.

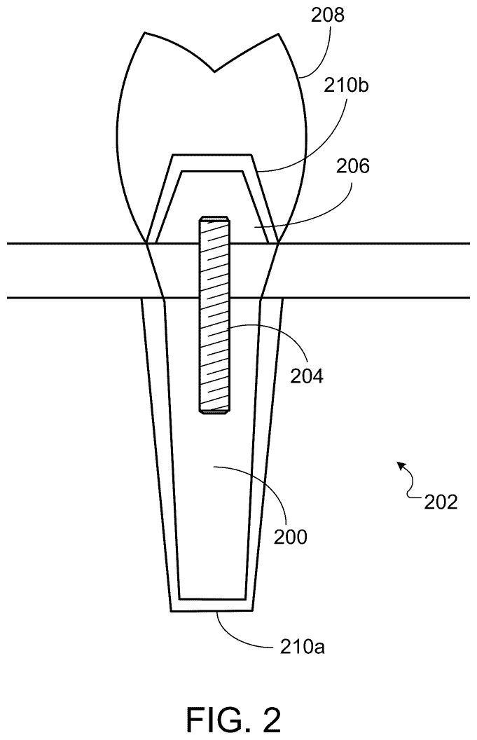

Bone cement with piezoelectric materials can also be used for dental implants. For example, referring to FIG. 2, a dental implant 200 is fastened to a jawbone 202. The implant 200 can be composed of a biocompatible metal (e.g., titanium) or ceramic. The implant 200 is fastened to the jawbone via a piezoelectric bone cement 210a including a piezoelectric material. The bone cement can be similar to the bone cement 108 described with respect to FIG. 1. The dental implant also includes a screw 204 that connects the implant to an abutment 206. The abutment 206 is also composed of a biocompatible metal (e.g., titanium) or ceramic. The abutment is attached to a crown 208, which can be composed of metal, porcelain, resin, or ceramic. The abutment is affixed to the crown via bone cement 210b. The bone cements 210a, 210b are similar to the bone cement 108 of FIG. 1, with one or both of the bone cements 210a, 210b including a piezoelectric material.

Piezoelectric bone cements can also be used for other types of orthopedic or dental implants other than those described above. For instance, bone cement can be used in knee implants, shoulder implants, elbow implants, or other suitable orthopedic implants. Additionally, bone cement can be used for other types of surgeries, such as osteoplasty.

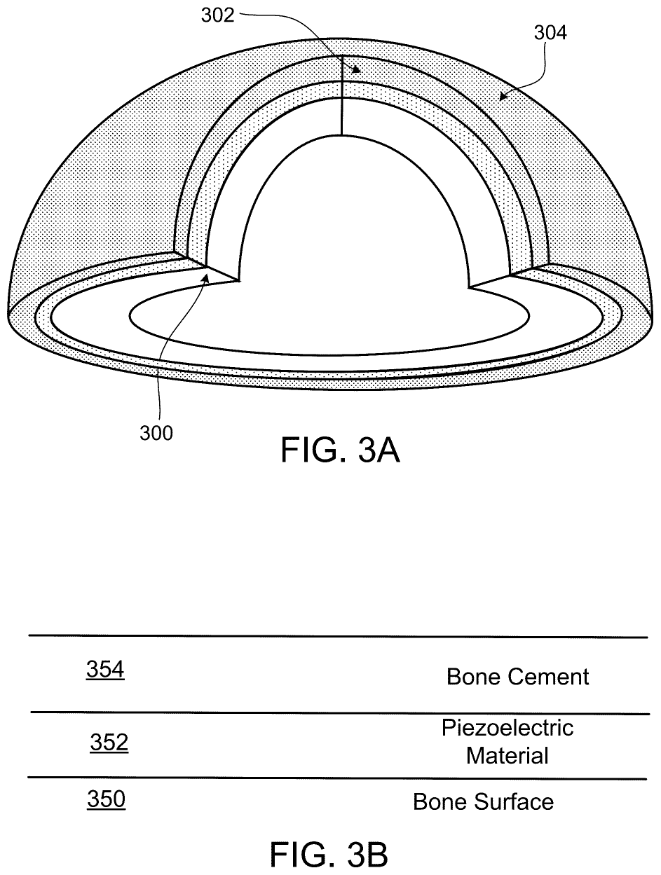

In some examples, piezoelectric bone cement can be applied to polymeric implants. Referring to FIG. 3A, in some examples, piezoelectric bone cement is applied to a polymeric acetabular cup implant 300 (e.g., an ultra-high molecular weight polyethylene (UHMWPE) implant. The polymer implant 300 is coated with a polymer layer 302, e.g., a layer of PMMA. The polymer-coated implant is cross-linked by application of a stimulus, e.g., irradiation by gamma radiation or other radiation, thermal processes, compression, or other suitable process. Cross-linked polyethylene can have improved characteristics (such as impact resistance and resistance to fracture) compared to traditional linear polyethylene. The cross-linked polymer-coated implant 300 is then coated in piezoelectric bone cement 304 for fixation to a bone surface. Piezoelectric bone cements can also be used for types of polymeric implants other than UHMWPE implants, such as polypropylene, polydimethylsiloxane, parylene, polyamide, polytetrafluoroethylene, etc.

Referring to FIG. 3B, in some examples, multiple layers of material are applied to a surface of a bone 350 to facilitate fixation of an implant to the surface of the bone 350. An inner layer 352 of piezoelectric material (e.g., barium titanate, hydroxyapatite, or a composite thereof) is applied directly to the bone 350. The direct contact between the bone 350 and the piezoelectric material of the inner layer 352 creates a strong bond. An outer layer 354 of bone cement is disposed on top of the layer 352 of piezoelectric material. The bone cement 354 can be similar to the bone cement 108 of FIG. 1. In some examples, the bone cement 354 is a piezoelectric bone cement; in some examples, the bone cement 354 does not include piezoelectric material. In some examples, the piezoelectric material of the inner layer 302 is coated with bone stem cells to further facilitate fixation between the bone cement and the bone.

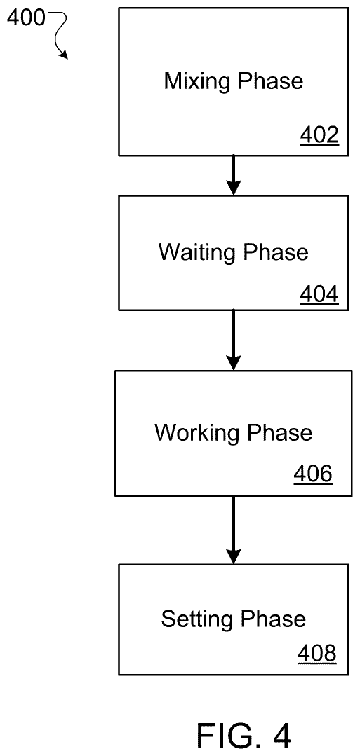

FIG. 4 illustrates a method 400 of using a piezoelectric bone cement to affix an implant, such as an orthopedic implant or a dental implant, to the surface of a bone.

First, the solid phase and the liquid phase of the piezoelectric bone cement are mixed 402 such that the powder of the solid phase is dispersed in the liquid phase. The solid phase includes a polymer powder (e.g., polymethyl methacrylate (PMMA), methyl methacrylate-styrene copolymer, polystyrene, methacrylate copolymer, or combinations thereof), an initiator (e.g., benzoyl peroxide), and a powder of a piezoelectric ceramic (e.g., barium titanate or hydroxyapatite). The liquid phase includes a liquid monomer (e.g., methyl methacrylate monomer), an accelerator (e.g., N, N-dimethyl para-toluidine or dimethyl para-toluidine), and a stabilizer (e.g., hydroquinone).

In an example, the liquid phase of the piezoelectric bone cement described here includes between 95 and 99 volume percent of the monomer (e.g., methyl methacrylate monomer), e.g., about 95 wt. %, about 96 vol. %, about 97 vol. %, about 98 vol. %, or about 99 vol. %. The liquid phase of the bone cement also includes between 1 and 4 volume percent of the accelerator (e.g., N,N,-dimethyl-p-toluidine), e.g., about 1 vol. %, about 2 vol. %, about 3 vol. %, or about 4 vol. %. The liquid phase of the bone cement also includes a stabilizer (e.g., hydroquinone), e.g., between 50 and 100 parts per million (ppm), e.g., between 60 and 90 ppm.

In an example, the solid phase of the piezoelectric bone cement described here includes between 85 and 95 weight percent of a powder of a polymer, such as a powder of PMMA methylmethacrylate-styrene copolymer, polystyrene, methacrylate copolymer, or another polymer, or combinations of two or more polymers. For instance, the solid phase can include between 10 and 20 wt. % of PMMA powder and between 70 and 80 wt. % of methyl methyacrylate-styrene copolymer. The solid phase also includes between 5 and 15 weight percent of a powder of a piezoelectric ceramic (e.g., piezoelectric barium titanate), e.g., between 7 and 10 wt. %. The solid phase also includes an initiator (e.g., dibenzoyl peroxide).

After mixing 402, the piezoelectric bone cement goes through a waiting phase 404. In this phase, the components of the bone cement are allowed to interact and start to solidify, e.g., through radical polymerization. The solid phase and liquid phase of the piezoelectric bone cement can be mixed together in a ratio of 15-25 mL of the liquid phase and 30-50 g of the solid phase. The composition and ratio of the solid and liquid phases can be selected such that the piezoelectric bone cement has a mixing time of between 3 and 10 minutes and a setting time of between 5 and 15 minutes, and achieves a maximum exotherm at 80-100° C. and a minimum intrusion of 1-3 mm.

Before the bone cement hardens completely, the bone cement enters the working phase 406. During the working phase 404, the piezoelectric bone cement is applied to an implant or bone surface. In some examples, prior to application of the bone cement to a bone surface, a powder of a piezoelectric ceramic (e.g., a powder of barium titanate, hydroxyapatite, or a composite thereof). is applied directly to the bone surface. The piezoelectric ceramic can be precoated in bone stem cells. The piezoelectric bone cement is applied to the surface of the bone on which the powder of the piezoelectric ceramic has already been applied. In some embodiments, the piezoelectric materials are applied directly to the implant or bone prior to the mixing phase 400 and the solid and liquid phase of the piezoelectric bone cement are mixed after both have been applied to the implant or bone.

After the working phase 404, the method continues to the setting phase 406, in which the bone cement is allowed to cure completely, affixing the implant to the bone. In some example, a stimulus, such as heat or light, is applied to facilitate curing. In some examples, the bone cement cures spontaneously. The result is a polymeric matrix, e.g., a matrix of PMMA methylmethacrylate-styrene copolymer, polystyrene, methacrylate copolymer, or another polymer, in which particles of a piezoelectric ceramic (e.g., barium titanate, hydroxyapatite, or a composite thereof) is dispersed. The composition and ratio of the solid and liquid phases can be selected such that the piezoelectric bone cement, after setting, has a compressive strength of at least 7 Pa, e.g., between 7 and 15 Pa; a maximum indentation of 0.2 mm, e.g., 0-0.2 mm; a minimum recovery of 60%, e.g., 60-100%; a maximum water sorption of 1 mg/mL, e.g., 0-1 mg /L; and a maximum water solubility of 0.05 mg/mL, e.g., 0-0.05 mg/mL.

Piezoelectric materials can also be employed to promote cell growth in cell culture dishes. Cell culture dishes are disposable or reusable shallow containers specifically designed to support the growth and propagation of cells in culture.

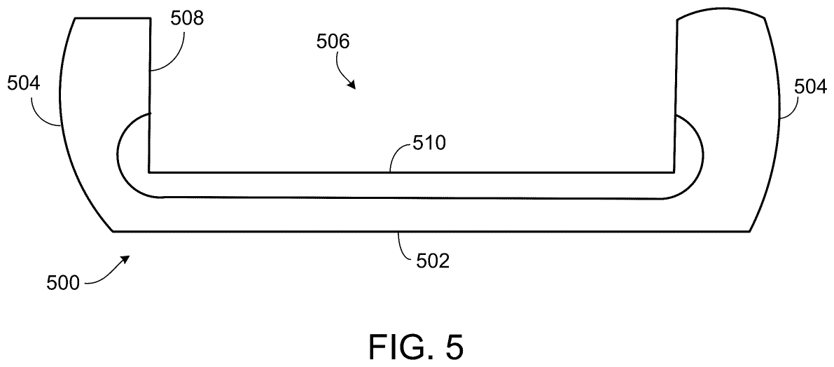

FIG. 5 illustrates an example piezoelectric cell culture dish 500. The piezoelectric cell culture dish 500 includes a base 502 and walls 504 connected to the base to define an interior 506. In some examples, the inner surfaces 508 of the walls 504, base 502, or both are treated to encourage adhesion and/or growth of anchor-dependent cells. Surface treatment can include texturing (e.g., by sand blasting, electron discharge, etc.), wetting, or both. In some examples, the inner surfaces 508 of the walls 504 and base 502 are untreated, e.g., to support a culture of suspension cells.

The piezoelectric cell culture dishes described here include piezoelectric materials, such as piezoelectric ceramics, on the inner surfaces 508 of the walls 504, base 502, or both. In the example of FIG. 5, a layer 510 of piezoelectric material is embedded in the walls 504 and base 502, and the walls 504, base 502, or both are themselves a non-piezoelectric material, such as glass, polymer, or ceramic. In some examples, a coating of a piezoelectric material is disposed on the walls 504, base 502, or both to form the inner surface, and the walls 504, base 502, or both are themselves a non-piezoelectric material. In some examples, the walls 504, base 502, or both are formed entirely from a piezoelectric material. Examples of piezoelectric materials that can be used with the cell culture dishes described here include quartz, barium titanate (BaTiO₃), polyvinylidene fluoride (PVDF), hydroxyapatite, or other piezoelectric materials that are compatible with the cell culture environment.

Piezoelectric cell culture dishes can stimulate cell and tissue growth because mechanical energy provided to the dishes is converted to electrical energy by the piezoelectric material. The electrical energy can promote cell or tissue growth. Stimulation can be provided, e.g., by chemicals, light, electromagnetic fields, etc.

In some examples, some or all of the piezoelectric material of a piezoelectric cell culture dish is a negative Poisson's ratio material (referred to as an “NPR material” or an “auxetic material”) or a material that is a composite of an NPR material and a material with a positive Poisson's ratio. An NPR material is a material that has a Poisson's ratio that is less than zero, such that when the material experiences a positive strain along one axis (e.g., when the material is stretched), the strain in the material along the two perpendicular axes is also positive (e.g., the material expands in cross-section). Conversely, when the material experiences a negative strain along one axis (e.g., when the material is compressed), the strain in the material along a perpendicular axis is also negative (e.g., the material compresses along the perpendicular axis). By contrast, a material with a positive Poisson's ratio (a “PPR material”) has a Poisson's ratio that is greater than zero. When a PPR material experiences a positive strain along one axis (e.g., when the material is stretched), the strain in the material along the two perpendicular axes is negative (e.g., the material compresses in cross-section), and vice versa.

In some examples, a piezoelectric material can be formed into an NPR material by forming nanoscale or microscale structures, such as spheres or tubules, with the piezoelectric material. The nano-or micro-structured piezoelectric NPR material can be provided as a coating on an inner surface of the cell culture dish, can be embedded into the material of the walls or base, or can constitute the entirety of the walls or base.

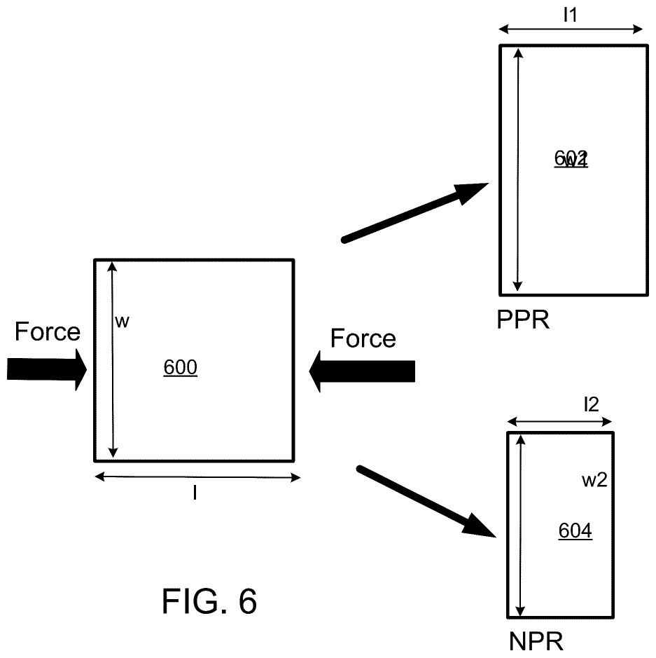

Materials with negative and positive Poisson's ratios are illustrated in FIG. 6, which depicts a hypothetical two-dimensional block of material 600 with length l and width w.

If the hypothetical block of material 600 is a positive Poisson's ratio (PPR) material, when the block of material 600 is compressed along its width w, the material deforms into the shape shown as block 602. The width w1 of block 602 is less than the width w of block 600, and the length l1 of block 602 is greater than the length l of block 600: the material compresses along its width and expands along its length.

By contrast, if the hypothetical block of material 600 is an NPR material, when the block of material 600 is compressed along its width w, the material deforms into the shape shown as block 604. Both the width w2 and the length l2 of block 604 are less than the width w and length l, respectively, of block 600: the material compresses along both its width and its length.

NPR materials can be foams, such as polymeric foams, ceramic foams, metallic foams, or combinations thereof. A foam is a multi-phase composite material in which one phase is gaseous and the one or more other phases are solid (e.g., polymeric, ceramic, or metallic). Foams can be closed-cell foams, in which each gaseous cell is sealed by solid material; open-cell foams, in which the each cell communicates with the outside atmosphere; or mixed, in which some cells are closed and some cells are open.

An example of an NPR foam structure is a re-entrant structure, which is a foam in which the walls of the cells are concave, e.g., protruding inwards toward the interior of the cells. In a re-entrant foam, compression applied to opposing walls of a cell will cause the four other, inwardly directed walls of the cell to buckle inward further, causing the material in cross-section to compress, such that a compression occurs in all directions. Similarly, tension applied to opposing walls of a cell will cause the four other inwardly directed walls of the cell to unfold, causing the material in cross-section to expand, such that expansion occurs in all directions.

NPR foams can have a Poisson's ratio of between −1 and 0, e.g., between −0.8 and 0, e.g., −0.8, −0.7, −0.6, −0.5, −0.4, −0.3, −0.2, or −0.1. NPR foams can have an isotropic Poisson's ratio (e.g., Poisson's ratio is the same in all directions) or an anisotropic Poisson's ratio (e.g., Poisson's ratio when the foam is strained in one direction differs from Poisson's ratio when the foam is strained in a different direction).

An NPR foam can be polydisperse (e.g., the cells of the foam are not all of the same size) and disordered (e.g., the cells of the foam are randomly arranged, as opposed to being arranged in a regular lattice). An NPR foam can have a characteristic dimension (e.g., the size of a representative cell, such as the width of the cell from one wall to the opposing wall) ranging from 0.1 μm to about 3 mm, e.g., about 0.1 μm, about 0.5 μm, about 1 μm, about 10 μm, about 50 μm, about 100 μm, about 500 μm, about 1 mm, about 2 mm, or about 3 mm.

Examples of polymeric foams include thermoplastic polymer foams (e.g., polyester polyurethane or polyether polyurethane); viscoelastic elastomer foams; or thermosetting polymer foams such as silicone rubber. Examples of metallic foams include metallic foams based on copper, aluminum, or other metals, or alloys thereof.

NPR-PPR composite materials are composites that include both regions of NPR material and regions of PPR material. NPR-PPR composite materials can be laminar composites, matrix composites (e.g., metal matrix composites, polymer matrix composites, or ceramic matrix composites), particulate reinforced composites, fiber reinforced composites, or other types of composite materials. In some examples, the NPR material is the matrix phase of the composite and the PPR material is the reinforcement phase, e.g., the particulate phase or fiber phase. In some examples, the PPR material is the matrix phase of the composite and the NPR material is the reinforcement phase.

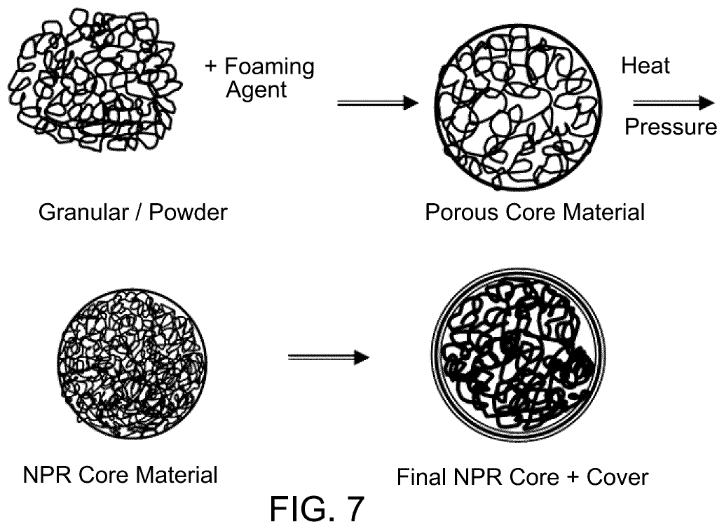

FIG. 7 illustrates an example method of making a core formed of an NPR material. A granular or powdered material, such as a polymer material (e.g., a rubber) is mixed with a foaming agent to form a porous material 50. The porous material 50 is placed into a mold 52. Pressure is applied to compress the material 50 and the compressed material is heated to a temperature above its softening point. The material is then allowed to cool, resulting in an NPR foam 54. The NPR foam 54 is covered with an outer layer 56, such as a polymer layer, and heat and pressure is applied again to cure the final material into a core 58.

Other embodiments are within the scope of the following claims.

What is claimed is:

1. A cell culture dish comprising:
a base; and
walls connected to the base to define an interior of the cell culture dish, wherein at least a portion of an interior surface of the base comprises a piezoelectric material.

2. The cell culture dish of claim 1, wherein the piezoelectric material comprises quartz.

3. The cell culture dish of claim 1, wherein the piezoelectric material comprises barium titanate.

4. The cell culture dish of claim 1, wherein the piezoelectric material comprises polyvinylidene fluoride.

5. The cell culture dish of claim 1, wherein the interior surface of the base is textured to promote cell growth.

6. The cell culture dish of claim 1, comprising a coating of the piezoelectric material disposed on the base, and wherein the coating forms the interior surface of the base.

7. The cell culture dish of claim 1, wherein the piezoelectric material comprises a negative Poisson's ratio (NPR) material, in which the NPR material has a Poisson's ratio of between 0 and −1.

8. The cell culture dish of claim 7, wherein the NPR material comprises microspheres of the piezoelectric material.

9. The cell culture dish of claim 7, wherein the NPR material comprises microtubules of the piezoelectric material.

10. A method of making a cell culture dish, the method comprising:
forming at least a portion of an interior surface of a base of a cell culture dish from a piezoelectric material;
wherein the cell culture dish comprises the base and walls connected to the base to define an interior of the cell culture dish.

11. The method of claim 10, wherein the piezoelectric material comprises quartz.

12. The method of claim 10, wherein the piezoelectric material comprises barium titanate.

13. The method of claim 10, wherein the piezoelectric material comprises polyvinylidene fluoride.

14. The method of claim 10, the method comprising forming the piezoelectric material into a negative Poisson's ratio (NPR) material, in which the NPR material has a Poisson's ratio of between 0 and −1, and in which the portion of the interior surface of the base of the cell culture dish is formed of the piezoelectric NPR material.

15. The method of claim 10, wherein forming the at least a portion of the interior surface from the piezoelectric material comprises disposing a coating of the NPR material on the base such that the coating forms the interior surface of the base.

16. The method of claim 14, wherein forming the piezoelectric material into the NPR material comprises forming microspheres of the piezoelectric material.

17. The method of claim 14, wherein forming the piezoelectric material into the NPR material comprises forming microtubules of the piezoelectric material.

18. The method of claim 10, the method comprising texturing the interior surface of the base to promote cell growth.

19. The method of claim 18, wherein texturing the interior surface comprises sand blasting, electron discharge texturing, or a combination thereof.

20. The method of claim 10, the method comprising wetting the interior surface of the base to promote cell growth.
